(19) United States
(12) Patent Application Publication
(10) Pub. No.: US 2025/0352258 A1
(43) Pub. Date: Nov. 20, 2025

(54) ENERGY TREATMENT MEDICAL DEVICES AND ENERGY TREATMENT METHODS

(71) Applicant: INTUITIVE SURGICAL OPERATIONS, INC., Sunnyvale, CA (US)

(72) Inventors: Timothy O'Brien, Sunnyvale, CA (US); Ryan C. Abbott, San Jose, CA (US); Erasmo Lopez Calleros, Fremont, CA (US)

(21) Appl. No.: 19/211,762

(22) Filed: May 19, 2025

Related U.S. Application Data
(60) Provisional application No. 63/649,527, filed on May 20, 2024.

(51) Int. Cl. A61B 18/00; A61B 90/00; A61B 90/90; G16H 40/63

(52) U.S. Cl. CPC A61B 18/00; A61B 90/03; A61B 90/90; G16H 40/63; A61B 2018/00208; A61B 2018/00214; A61B 2018/00702; A61B 2018/00773; A61B 2090/034

(57) ABSTRACT

Medical devices and cartridges include a rotary drive portion and a plurality of arms movable by rotation of the rotary drive portion. An electrode is coupled to each of the arms. Medical systems and methods include a base and a plurality of cartridges configured to be releasably attached to the base. The medical systems and methods include an identification of a selected cartridge and a determination of a set of parameters for an energy treatment corresponding to the selected cartridge.

### CROSS-REFERENCE TO RELATED APPLICATION

This application claims the priority benefit of U.S. Provisional Application No. 63/649,527, filed May 20, 2024, which is hereby incorporated by reference herein in its entirety.

### FIELD

Disclosed embodiments relate to medical devices, and more specifically, to energy treatment medical devices.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, physicians may insert minimally invasive medical instruments (including surgical, diagnostic, therapeutic, and/or biopsy instruments) to reach a target tissue location. In minimally invasive robotically-assisted surgery, there is a time associated with switching out different instruments and/or end effectors. Reducing this time can advantageously reduce the time a patient is under general anesthesia.

### SUMMARY

The following presents a simplified summary of various examples described herein and is not intended to identify key or critical elements or to delineate the scope of the claims.

In some examples, a medical device is described herein that includes a body including a rotary drive portion, a plurality of arms, and a plurality of electrodes. The plurality of arms are pivotably coupled to the body to be movable by rotation of the rotary drive portion of the body between a retracted state extending along the body and an extended state at least partially extending outwardly from the body and each of the plurality of arms have one of the plurality of electrodes coupled thereto. In the retracted state, the arms extend along the body closer towards the body (e.g., radially closer or closer relative to a longitudinal axis of the device) than in the extended state. In some embodiments, the arms may abut against the body in the retracted state or the arms may be spaced apart from the body.

In further examples, the medical device includes an end effector base and a cartridge releasably coupled to the end effector base, where the cartridge includes the body, the plurality of arms, and the plurality of electrodes.

In further examples, the medical device includes a rotary drive output coupled to and configured to drive rotation of the rotary drive portion of the body. The medical device can include an end effector base coupled to the body and a rotary drive received within the end effector base, the rotary drive output comprising an output shaft of the rotary drive. The medical device can include an elongate instrument and a torque shaft extending within the elongate instrument, wherein the body is coupled to a distal end of the elongate instrument and the torque shaft is rotatably coupled between the rotary drive output and the rotary drive portion of the body.

Any of the above examples can include one or more of the following aspects: the drive portion includes a drive gear and interior ends of the respective plurality of arms comprise a driven gear configured to mesh with and be driven by drive gear; the plurality of arms are pivotable to position the plurality of electrodes at two or more predetermined positions; the plurality of arms are pivotable to position the plurality of electrodes at three or more predetermined positions; at least one of the plurality of electrodes has a solid cross-section; at least one of the plurality of electrodes defines a lumen extending therethrough for fluid delivery; at least one of the plurality of electrodes comprises a surface electrode; the plurality of electrodes have asymmetric lengths; the medical device includes a central electrode coupled to the body radially inwardly of the plurality of electrodes coupled to the plurality of arms; rotation of the rotary drive portion of the body causes the plurality of arms to move in concert; the plurality of arms are two arms and the plurality of electrodes are two electrodes; the plurality of arms comprises three arms and the plurality of electrodes comprises three electrodes; the plurality of arms comprises four arms and the plurality of electrodes comprises four electrodes; or the medical device includes an electrode tip cover movable to selectively expose distal tips of the plurality of electrodes.

In some examples, a medical system is described that includes an end effector having a central longitudinal axis. The end effector includes a base, and a plurality of cartridges configured to be releasably attached to the base, each of the plurality of cartridges including a plurality of arms, each arm of the plurality of arms having an electrode coupled thereto and being pivotable to dispose the electrode at a plurality of distances relative to the central longitudinal axis of the end effector, the plurality of cartridges having at least one characteristic different from one another. The medical system further includes an energy source and a control system configured to: identify a selected one of the plurality of cartridges to be used for an energy treatment, determine a set of parameters for the energy treatment corresponding to the selected one of the plurality of cartridges, and supply energy to the end effector from the energy source to perform the energy treatment based on the set of parameters.

In further examples, the set of parameters include one or more of: total length of the electrodes, length of an exposed portion of the electrodes, length of an insulated portion of the electrodes, electrode spacing, and a number of electrodes; the control system is configured to receive an identification of the selected one of the plurality of cartridges; the energy treatment includes a plurality of predetermined doses; and/or the medical system includes a motor, the cartridge including a drive portion coupled to the motor when attached to the base of the end effector to be rotated thereby, and wherein the control system is configured to control operation of the motor to pivot the plurality of arms and dispose the electrodes at a desired distance relative to the central longitudinal axis of the cartridge.

Any of the above examples can include one or more of the following aspects: the end effector is a drop-in probe; the end effector is a distal end of an elongate instrument; the end effector includes an electrode tip cover selectively movable to expose distal tips of the electrodes for treatment; the control system is configured to supply at least one of bipolar or monopolar energy to perform the energy treatment; or the control system is further configured to use one or more of the electrodes for sensing one or more patient attributes.

In some examples, an energy treatment method is described that includes identifying, by a control system, a selected cartridge from a plurality of cartridges having at least one characteristic different from one another and configured to releasably couple to a base of an end effector, the plurality of cartridges each having a plurality of arms, each arm of the plurality of arms having an electrode coupled thereto, coupling the selected cartridge to the base of the end effector, pivoting the plurality of arms of the selected cartridge from a retracted state to an extended state to dispose the electrodes at a desired distance relative to a central longitudinal axis of the end effector, determining, by the control system, a set of parameters for an energy treatment corresponding to the selected cartridge, and supplying, by the control system, energy to the end effector to perform the energy treatment based on the set of parameters.

In further examples, the set of parameters comprise one or more of: total length of the electrodes, length of an exposed portion of the electrodes, length of an insulated portion of the electrodes, electrode spacing, and a number of electrodes and/or identifying the selected cartridge from the plurality of cartridges includes receiving an identification of the selected cartridge from the plurality of cartridges.

Any of the above examples can include one or more of the following aspects: supplying, by the control system, energy to the end effector to perform the energy treatment includes supplying a plurality of predetermined doses of energy to the end effector to perform the energy treatment; pivoting the plurality of arms of the selected cartridge includes controlling operation of a motor to pivot the plurality of arms and dispose the electrodes at a desired distance relative to the central longitudinal axis of the end effector; the method includes moving an electrode tip cover coupled to the cartridge to expose distal tips of the electrodes for treatment; supplying energy to the end effector to perform the energy treatment includes supplying at least one of bipolar or monopolar energy to the end effector to perform the energy treatment; or the method includes sensing one or more patient attributes with one or more of the electrodes.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1 is a schematic diagram for a robotically-assisted manipulator system, according to some examples.

FIG. 2A is a schematic diagram of an instrument system according to examples described herein.

FIG. 2B illustrates a distal portion of the instrument system of FIG. 2A with an extended example of an instrument according to examples described herein.

FIG. 3A is a perspective view of a medical device according to examples described herein.

FIG. 3B is a perspective view of a distal end portion of the medical device of FIG. 3A.

FIG. 4A is a schematic diagram of a medical system according to examples described herein.

FIG. 4B is a front perspective view of a medical device of the medical system of FIG. 4A showing arms in a retracted state according to examples described herein.

FIG. 4C is a front perspective view of the medical device of FIG. 4B showing the arms in an extended state.

FIG. 4D is a perspective view of a medical device of the medical system of FIG. 4A having base and a cartridge having electrodes and a cover received on the electrodes according to examples described herein.

FIG. 4E is a perspective view of the medical device of FIG. 4D showing the cover slid to expose the electrodes.

FIG. 5A is a front perspective view of a medical device showing arms in a retracted state according to examples described herein.

FIG. 5B is a front perspective view of the medical device of FIG. 5A showing the arms in an extended state according to examples described herein.

FIG. 6 is a schematic diagram of a medical system according to examples described herein.

FIG. 7 is a schematic diagram of a method for energy treatment according to some examples described herein.

FIG. 8A is a perspective view of a first example medical device for a medical system according to examples described herein.

FIG. 8B is a perspective view of a second example medical device for a medical system according to examples described herein.

FIG. 8C is a schematic diagram of a medical system suitable for the medical devices of FIGS. 8A and 8B according to examples described herein.

FIG. 8D is a perspective view of a third example medical device for a medical system according to examples described herein.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

In the following description, specific details are set forth describing some embodiments consistent with the present disclosure. Numerous specific details are set forth in order to provide a thorough understanding of the embodiments. It will be apparent, however, to one skilled in the art that some embodiments may be practiced without some or all of these specific details. The specific embodiments disclosed herein are meant to be illustrative but not limiting. One skilled in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional. In some instances, well known methods, procedures, components, and circuits have not been described in detail so as not to unnecessarily obscure aspects of the embodiments.

This disclosure describes various instruments and portions of instruments in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (e.g., one or more degrees of rotational freedom such as, roll, pitch, and yaw). As used herein, the term “pose” refers to the position of an object or a portion of an object in at least one degree of translational freedom and to the orientation of that object or portion of the object in at least one degree of rotational freedom (e.g., up to six total degrees of freedom). As used herein, the term “shape” refers to a set of poses, positions, and/or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

Medical devices, medical systems, and related methods are described herein directed to a medical device (e.g., an end effector) with electrodes coupled to pivotable arms that can be delivered to a treatment site in a compact configuration and subsequently pivoted to an extended configuration to increase a treatment volume for energy-based therapy. In the extended configuration, the pivotable arms are spaced apart further from each other than in the compact configuration. The end effector provides consistent, parallel electrode-to-electrode positioning, while also allowing the electrode-to-electrode spacing to be selectively changed (e.g., increased or decreased based on an amount of deployment of the pivotable arms). In some versions, the end effector can be a drop-in component (e.g., a tool that can be delivered to a treatment site and grasped/positioned by other surgical instruments to provide treatment). In other versions, the end effector can be a component of an elongated surgical instrument, for example an instrument including an elongated torque/drive shaft extending proximally to a backend housing. The backend housing may include a manual control interface for a user to manually actuate the instrument, or a robotic interface to couple to a robotic system, or a combination of a manual control interface and a robotic interface.

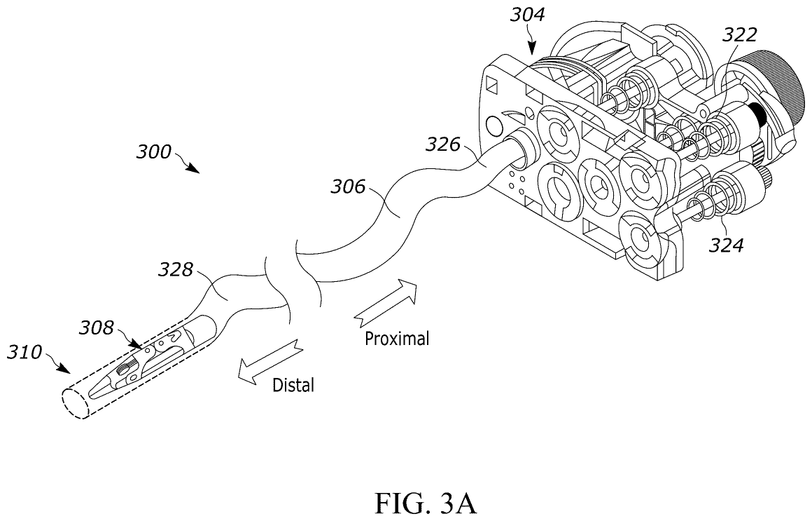

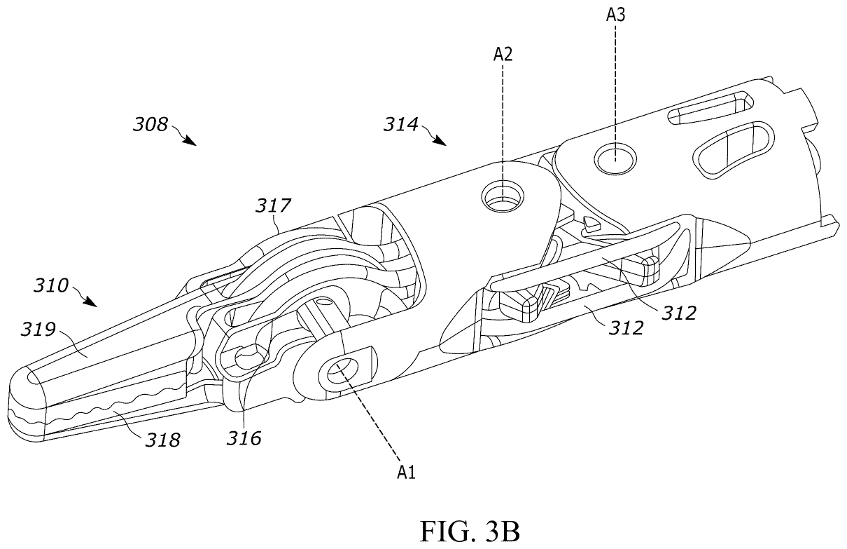

In the drop-in version, an energy treatment end effector or medical device includes a motor, a body having a drive portion coupled to the motor to be rotated by an output of the motor, and a plurality of arms, each of the plurality of arms having at least one electrode coupled thereto. The plurality of arms are pivotably coupled to the body and are movable by rotation of the drive portion of the body between a retracted state and an extended state. extending along the body and an extended state at least partially extending outwardly from the body. In the retracted state, the arms extend along the body closer towards the body (e.g., radially closer or closer relative to a longitudinal axis of the device) than in the extended state. In some embodiments, the arms may abut against the body in the retracted state or the arms may be spaced apart from the body. So configured, the plurality of arms can be selectively pivoted via operation of the motor to dispose the electrodes at different distances relative to a longitudinal axis of the end effector to provide a desired treatment volume. In the elongated surgical instrument version (which may be similar to the medical device 300 depicted in FIG. 3A), the motor can be a component of a robotic system. The robotic system has motor outputs that can be operably coupled to robotic inputs at a backend housing of the surgical instrument to transmit forces and torques to the surgical instrument. The backend housing of the surgical instrument includes a force transmission mechanism (which may be similar to force transmission mechanism 304) coupled to a proximal end of the elongated torque/drive shaft to transmit forces/torques to the distal end of the instrument. In the elongated surgical instrument version, the body including the drive portion, arms, and electrodes, is disposed at the distal end of the elongated torque/drive shaft. The robotic system may additionally include one or more energy sources (e.g., power supplies) and a control system to electrically activate and control energy delivery to the electrodes of the elongated surgical instrument when coupled to the robotic system.

According to some examples, the end effector includes a base and a releasable cartridge, where the cartridge includes the body, the plurality of arms, and the electrodes. This functionality allows a user to select a cartridge having characteristics for a desired energy treatment. For example, the cartridges can have a variety of one or more of: number of arms/electrodes (e.g., 2, 3, 4, etc.), total length of the electrodes, length of the exposed portion of the electrodes that can apply energy to tissue, length of the insulated portion of the electrodes, type of electrodes (e.g., surface electrodes, hollow needle electrodes, and/or solid needle electrodes), and so forth. The end effector can be a drop-in device configured to be manipulated and used via a tool coupled to instrument at a treatment location or can be coupled to an instrument (e.g., an elongate device) to be the end effector of the instrument. The end effector can also include a cover or cap configured to be removably disposed over tips of the electrodes to allow the end effector to be safely delivered to a treatment location and maneuvered at the treatment location prior to treatment.

A medical system and method include an end effector having a central longitudinal axis, an energy source, and a control system configured to supply energy to the end effector from the energy source to perform an energy treatment. The end effector includes a base and a plurality of cartridges configured to be releasably attached to the base, where the plurality of cartridges have at least one characteristic different from one another. Each of the plurality of cartridges includes a plurality of arms, each arm has at least one electrode coupled thereto and is pivotable about an inner end to dispose the electrode at a plurality of different distances relative to the central longitudinal axis of the end effector. In some examples, the end effector can have a configuration as described above. The control system is configured to identify a selected one of the plurality of cartridges to be used for an energy treatment, determine a set of parameters for the energy treatment corresponding to the selected one of the plurality of cartridges, and supply energy to the end effector from the energy source to perform the energy treatment based on the set of parameters.

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

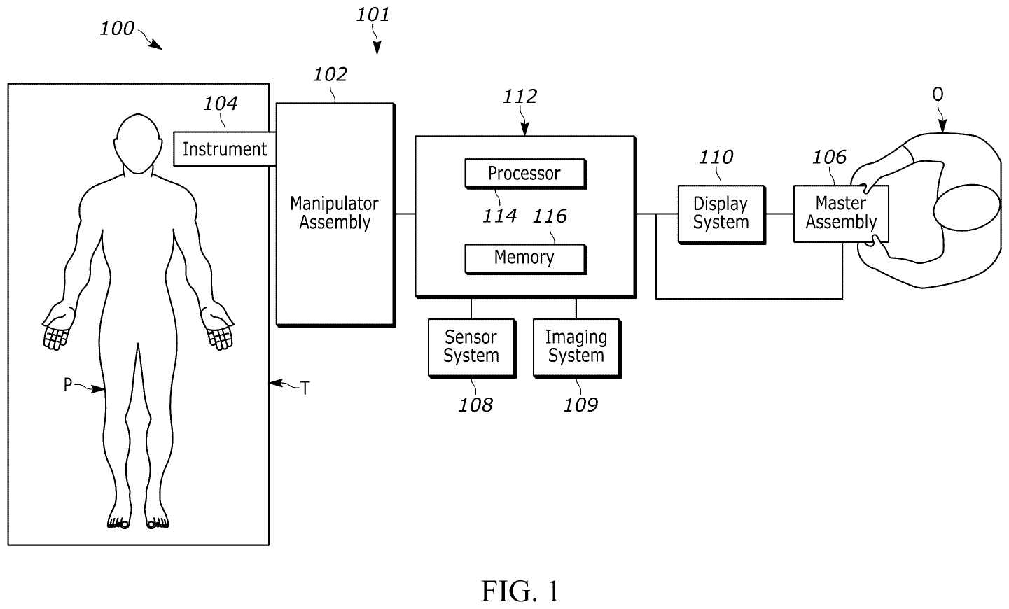

FIG. 1 illustrates an embodiment of a robotically-assisted manipulator system for use with the medical devices described herein. The manipulator system can be used, for example, in surgical, diagnostic, therapeutic, biopsy, or non-medical procedures, and is generally indicated by the reference numeral 100. As shown in FIG. 1, a robotically-assisted manipulator system 100 can include one or more manipulator assemblies 102 for operating one or more medical instrument systems 104 in performing various procedures on a patient P positioned on a table T in a medical environment 101. For example, the manipulator assembly 102 can drive catheter or end effector motion, can apply treatment to target tissue, and/or can manipulate control members. The manipulator assembly 102 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 106, which can be inside or outside of the medical environment 101, generally includes one or more control devices for controlling manipulator assembly 102. Manipulator assembly 102 supports medical instrument system 104 and can optionally include a plurality of actuators or motors that drive inputs on medical instrument system 104 in response to commands from a control system 112. The actuators can optionally include drive systems that when coupled to medical instrument system 104 can advance medical instrument system 104 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 102 can support various other systems for irrigation, treatment, or other purposes. Such systems can include fluid systems (including, for example, reservoirs, heating/cooling elements, pumps, and valves), generators, lasers, interrogators, and ablation components.

Robotically-assisted manipulator system 100 also includes a display system 110 for displaying an image or representation of the surgical site and medical instrument system 104 generated by an imaging system 109 which can include an imaging system, such as an endoscopic imaging system. Display system 110 and operator input system 106 can be oriented so an operator O can control medical instrument system 104 and operator input system 106 with the perception of telepresence. A graphical user interface can be displayable on the display system 110 and/or a display system of an independent planning workstation.

In some examples, the endoscopic imaging system components of the imaging system 109 can be integrally or removably coupled to medical instrument system 104. However, in some examples, a separate imaging device, such as an endoscope, attached to a separate manipulator assembly can be used with medical instrument system 104 to image the surgical site. The endoscopic imaging system 109 can be implemented as hardware, firmware, software, or a combination thereof which interact with or are otherwise executed by one or more computer processors, which can include the processors of the control system 112.

Robotically-assisted manipulator system 100 can also include a sensor system 108. The sensor system 108 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 104. The sensor system 108 can also include temperature, pressure, force, or contact sensors or the like.

Robotically-assisted manipulator system 100 can also include a control system 112. Control system 112 includes at least one memory 116 and at least one computer processor 114 for effecting control between medical instrument system 104, operator input system 106, sensor system 108, and display system 110. Control system 112 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 112 can optionally further include a virtual visualization system to provide navigation assistance to operator O when controlling medical instrument system 104 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 112 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

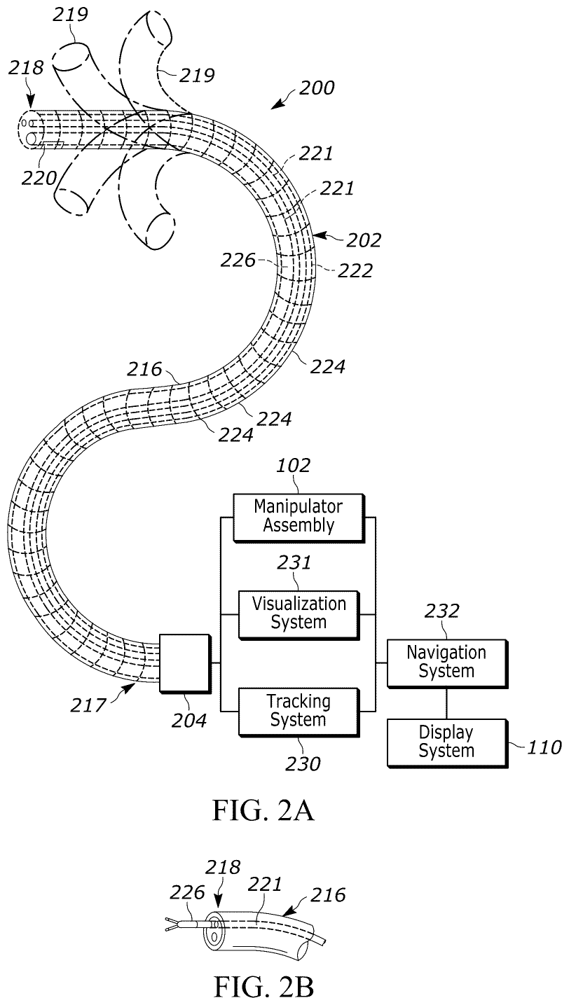

FIG. 2A shows a medical instrument system 200 according to some embodiments. In some embodiments, medical instrument system 200 can be used in an image-guided medical procedure. In some examples, medical instrument system 200 can be used for non-teleoperational exploratory procedures or in procedures involving traditional manually operated medical instruments, such as endoscopy. In some embodiments, medical instrument system 200 is interchangeable with, or a variation of, medical instrument system 104 of FIG. 1.

Medical instrument system 200 includes elongate flexible device 202, such as a flexible catheter or endoscope (e.g., gastroscope, bronchoscope), coupled to a drive unit 204. Elongate flexible device 202 includes a flexible body 216 having proximal end 217 and distal end, or tip portion, 218. In some embodiments, flexible body 216 has an approximately 14-20 mm outer diameter. Other flexible body outer diameters can be larger or smaller. Flexible body 216 can have an appropriate length to reach certain portions of the anatomy, such as the lungs, sinuses, throat, or the upper or lower gastrointestinal region, when flexible body 216 is inserted into a patient's oral or nasal cavity.

Medical instrument system 200 optionally includes a tracking system 230 for determining the position, orientation, speed, velocity, pose, and/or shape of distal end 218 and/or of one or more segments 224 along flexible body 216 using one or more sensors and/or imaging devices. The entire length of flexible body 216, between distal end 218 and proximal end 217, can be effectively divided into segments 224. Tracking system 230 can optionally be implemented as hardware, firmware, software, or a combination thereof which interact with or are otherwise executed by one or more computer processors, which can include the processors of control system 112 in FIG. 1.

Tracking system 230 can optionally track distal end 218 and/or one or more of the segments 224 using a shape sensor 222. In some embodiments, tracking system 230 can optionally and/or additionally track distal end 218 using a position sensor system 220, such as an electromagnetic (EM) sensor system. In some examples, position sensor system 220 can be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 216 includes one or more channels 221 sized and shaped to receive one or more medical instruments 226. In some embodiments, flexible body 216 includes two channels 221 for separate instruments 226, however, a different number of channels 221 can be provided. FIG. 2B is a simplified diagram of flexible body 216 with medical instrument 226 extended according to some embodiments. In some embodiments, medical instrument 226 can be used for procedures and aspects of procedures, such as surgery, biopsy, ablation, mapping, imaging, illumination, irrigation, or suction. Medical instrument 226 can be deployed through channel 221 of flexible body 216 and used at a target location within the anatomy. Medical instrument 226 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, transducers, sensors, imaging devices and/or the like. Medical instrument 226 can be advanced from the opening of channel 221 to perform the procedure and then retracted back into the channel when the procedure is complete. Medical instrument 226 can be removed from proximal end 217 of flexible body 216 or from another optional instrument port (not shown) along flexible body 216. The medical instrument 226 can be used with an image capture device (e.g., an endoscopic camera) also within the elongate flexible device 202. Alternatively, the medical instrument 226 can itself be the image capture device.

Medical instrument 226 can additionally house cables, linkages, or other actuation controls (not shown) that extend between its proximal and distal ends to controllably the bend distal end of medical instrument 226. Flexible body 216 can also house cables, linkages, or other steering controls (not shown) that extend between drive unit 204 and distal end 218 to controllably bend distal end 218 as shown, for example, by broken dashed line depictions 219 of distal end 218. In some examples, at least four cables are used to provide independent “up-down” steering to control a pitch motion of distal end 218 and “left-right” steering to control a yaw motion of distal end 218. In embodiments in which medical instrument system 200 is actuated by a robotically-assisted assembly, drive unit 204 can include drive inputs that removably couple to and receive power from drive elements, such as actuators, of the teleoperational assembly. In some embodiments, medical instrument system 200 can include gripping features, manual actuators, or other components for manually controlling the motion of medical instrument system 200. The information from tracking system 230 can be sent to a navigation system 232 where it is combined with information from visualization system 231 and/or the preoperatively obtained models to provide the physician or other operator with real-time position information.

FIGS. 3A and 3B are various views of a medical device 300, according to an embodiment. In some embodiments, the medical device 300 or any of the components therein are optionally parts of an instrument for a surgical system that performs surgical procedures, and which surgical system can include a manipulator unit, a series of kinematic linkages, a series of cannulas, or the like. The medical device 300 (and any of the instruments described herein) can be used in any suitable surgical system, such as the manipulator system 100 or the manipulator system 200 shown and described above. The medical device 300 can be used as the medical instrument 226 described above. As shown in FIG. 3A, the medical device 300 defines (or is included within) a distal boundary (or footprint) 302 that corresponds to a cannula size, or a size to fit within a working channel of an elongate flexible device (such as a flexible catheter or endoscope), or other size dictated by the surgical environment. The distal boundary 302 can be a cylindrical shape having any suitable nominal diameter (e.g., 8 mm, 5 mm, or any size therebetween). The medical device 300 includes a force transmission mechanism 304, a shaft 306, an optional distal wrist assembly 308, a distal end effector 310, and a set of tension elements 312 (which can be, for example, a cable, band, or the like).

The medical device 300 can include multiple tension elements 312. For example, in some embodiments, the medical device 300 can include two tension elements 312 with each tension element 312 having two segments extending along the shaft 306 of the instrument, thereby forming four proximal end portions. Referring to FIG. 3B, respective tension elements 312 can be routed through a wrist assembly 314 and wrapped about respective pulleys 316 or 317 of the tool members 318, 319. Each tension element 312 has two tension element segments along the shaft 306 with two proximal end portions that, when moved in opposite directions, can (among other things) cause rotation of the respective tool member 318 or 319 about the axis A1. This arrangement can be referred to as a “four cable” wrist and changing the pitch, yaw, or grip of the device 300 can be performed by manipulating the four proximal end portions of the tension elements 312 in a manner similar to that shown and described in U.S. Patent Publication No. 2020/0390430 incorporated herein by reference above. In other embodiments, the medical device 300 can include four separate tension elements 312 with two separate tension elements coupled to the pulley 316 of the tool member 318 and two separate tension elements coupled to the pulley 317 of the tool member 318, thereby creating four proximal tension element end portions. In some embodiments, the medical device 300 can include more than two or four tension elements 312 and more than four proximal tension element end portions. The tension elements 312 can be, for example, cables, bands, or the like that couple the force transmission mechanism 304 to the distal wrist assembly 308 and end effector 310. In some embodiments, the tension elements 312 can be constructed from a polymer.

The medical device 300 is configured such that movement of one or more of the tension elements 312 produces rotation of the end effector 310 about a first rotation axis A1 (see FIG. 3B, which functions as a yaw axis, the term yaw is arbitrary), rotation of the wrist assembly 308 about a second rotation axis A2 and/or optionally about a third rotation axis A3 (which functions as a pitch axis, the term pitch is arbitrary), a cutting rotation of the tool members of the end effector 310 about the first rotation axis A1, or any combination of these movements. Changing the pitch or yaw of the medical device 300 can be performed by manipulating the tension elements 312 in a similar manner as that described with reference to the device 2400 described in copending International Patent Application Serial No. PCT/US2022/039942, entitled “Surgical Instrument Cable Control and Routing Structures,” the disclosure of which is incorporated herein by reference in its entirety.

As shown in FIG. 3A, the proximal force transmission mechanism 304 includes a set of drive components such as capstans 322 and 324 that rotate or “wind” a proximal portion of any of the tension elements 312 to produce the desired tension element movement. In some embodiments, two proximal ends of a tension element 312, which are associated with opposing directions of a single degree of freedom, are connected to two independent drive capstans 322 and 324. This arrangement, which is generally referred to as an antagonist drive system, allows for independent control of the movement of (e.g., pulling in or paying out) each of the ends of the tension elements 312. The force transmission mechanism 304 produces movement of the tension elements 312, which operates to produce the desired articulation movements (pitch, yaw, or grip) at the wrist assembly 308 and end effector 310. Accordingly, the force transmission mechanism 304 includes components to move a first proximal end portion of the tension element 312 via the first capstan 322 in a first direction (e.g., a proximal direction) and to move a second proximal end portion of the tension element 312 via the second capstan 324 in a second opposite direction (e.g., a distal direction). The force transmission mechanism 304 can also move both proximal end portions of the tension element 312 in the same direction. In this manner, the force transmission mechanism 304 can maintain the desired tension within the tension elements 312.

In some embodiments, the force transmission mechanism 304 can include any of the assemblies or components described in International Patent Application Serial No. PCT/US2022/039942, entitled “Surgical Instrument Cable Control and Routing Structures,” the disclosure of which is incorporated herein by reference in its entirety. In other embodiments, however, any of the medical devices described herein can have the two ends of a tension elements wrapped about a single capstan. This alternative arrangement, which is generally referred to as a self-antagonist drive system, operates the two ends of the tension element using a single drive motor.

Moreover, although the force transmission mechanism 304 is shown as including capstans, in other embodiments, a force transmission mechanism can include one or more linear actuators that produce translation (linear motion) of a portion of the cables. Such force transmission mechanisms can include, for example, a gimbal, a lever, or any other suitable mechanism to directly pull (or release) an end portion of any of the cables. For example, in some embodiments, the proximal force transmission mechanism 304 can include any of the proximal force transmission mechanisms or components described in U.S. Patent Application Pub. No. US 2015/0047454 A1 (filed Aug. 15, 2014), entitled “Lever Actuated Gimbal Plate,” or U.S. Patent No. U.S. Pat. No. 6,817,974 B2 (filed Jun. 28, 2001), entitled “Surgical Tool Having Positively Positionable Tendon-Actuated Multi-Disk Wrist Joint,” each of which is incorporated herein by reference in its entirety.

The shaft 306 can be any suitable elongated shaft that is coupled to the force transmission mechanism 304 and to the optional wrist assembly 308 (when present) or the end effector 310. Specifically, the shaft 306 includes a proximal portion 326 that is coupled to the force transmission mechanism 304, and a distal portion 328 that is coupled to the optional wrist assembly 308 or to the end effector 310. The shaft 306 defines a passageway or series of passageways through which the tension elements 312 and other components (e.g., electrical wires, ground wires, or the like) can be routed from the force transmission mechanism 304 to the wrist assembly 308. In some embodiments, the shaft 306 can be a substantially rigid member, while in other embodiments, the shaft 306 can be a flexible member.

Other configurations of teleoperated manipulator systems are also contemplated, such as systems configured for multi-port or single-port procedures. For example, the embodiments described herein can be used with a da Vinci® Surgical System, such as the da Vinci X®, Xi®, or SP® Surgical Systems, all commercialized by Intuitive Surgical, Inc., of Sunnyvale, California.

In these examples, one or more instruments include shafts having a moveable end effector, endoscope, camera, or other sensing device at a distal end of the shaft, and can optionally include or exclude a wrist mechanism (not shown) to control the movement of the distal end. In these examples, the shafts can be partially or entirely rigid (e.g., not bendable/articulable). The instruments can be utilized through the cannula to directly access a treatment location, relying on actuation of end effector mechanisms to perform desired operations during a procedure.

In some approaches, the distal end portions of the instruments of these examples are received through a port structure (e.g., single or multi-port structure) to be introduced into the patient. Suitable port structures can include a cannula and an instrument entry guide inserted into the cannula. Individual instruments are inserted into the entry guide to reach a surgical site.

The manipulator systems described herein are not limited to the embodiments of FIGS. 1-3B, and various other teleoperated, computer-assisted manipulator configurations can be used with the embodiments described herein. The diameter or diameters of an instrument shaft and end effector are generally selected according to the size of the cannula with which the instrument will be used and depending on the surgical procedures being performed.

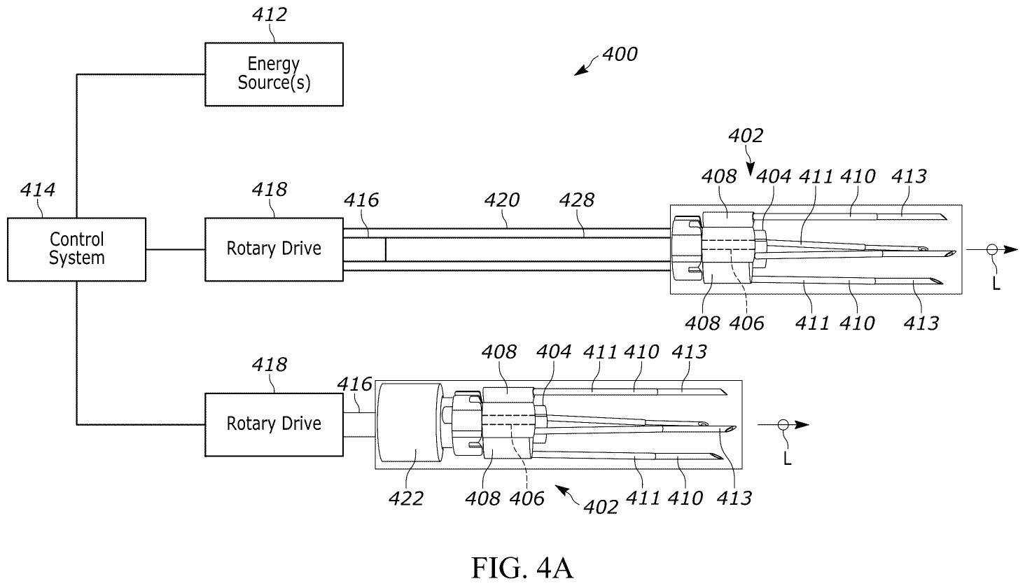

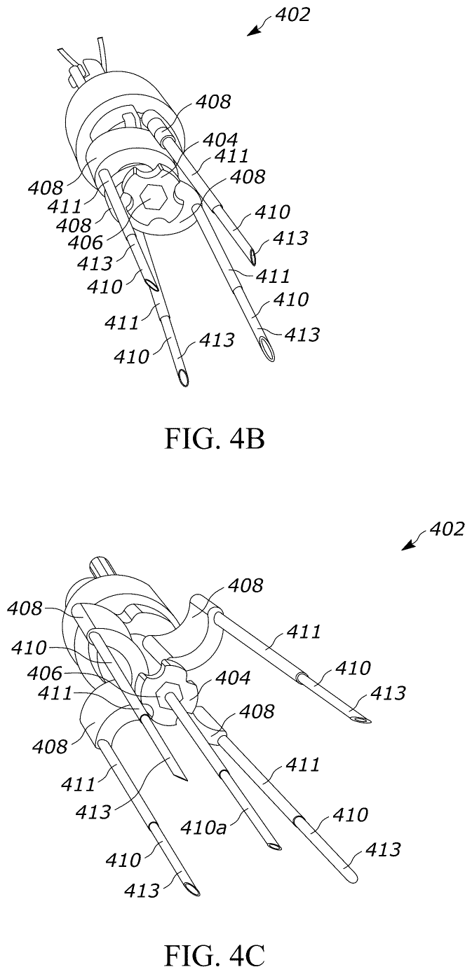

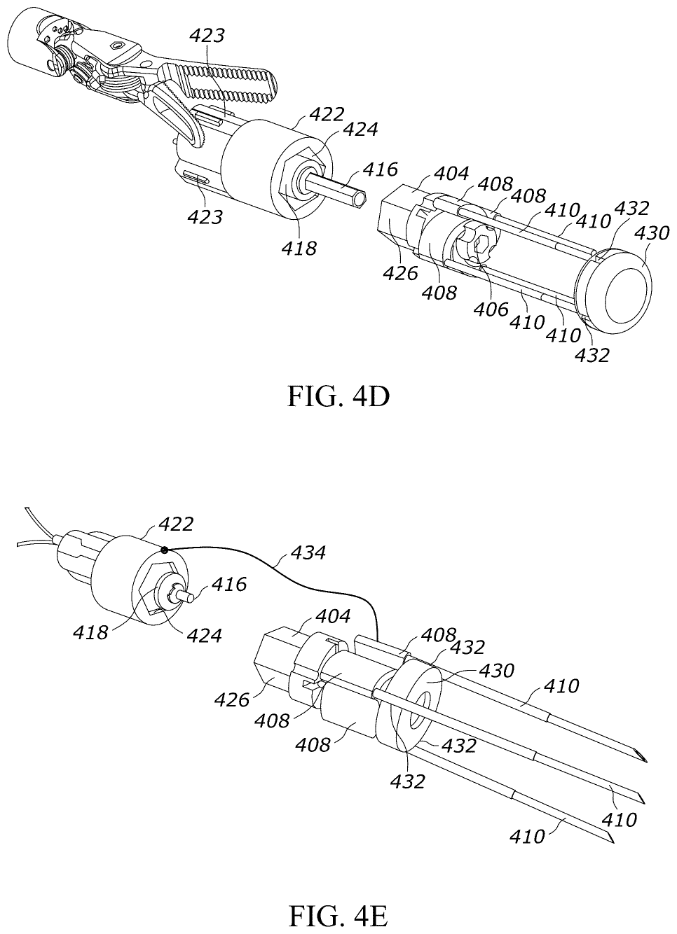

FIGS. 4A-4E show a medical system 400 and components thereof according to some embodiments. The medical system 400 includes a medical device 402, such as an end effector, capable of performing energy treatment (e.g., penetrating and/or surface energy treatment). The energy treatment can include a single dose or can include a plurality of predetermined doses. The medical device 402 includes a body 404 with a rotary drive portion 406 and a plurality of arms 408 pivotably coupled to the body 404 and movable by rotation of the rotary drive portion 406. The arms 408 are pivotable between a retracted state (FIG. 4B) extending along the body 404 and one or more extended states (FIG. 4C) at least partially extending outwardly from the body 404. In the retracted state, the arms extend along the body closer towards the body (e.g., radially closer or closer relative to a longitudinal axis of the device) than in the extended state. In some embodiments, the arms may abut against the body in the retracted state or the arms may be spaced apart from the body. As shown, each of the arms 408 has an electrode 410 coupled thereto, such that pivoting of the arms 408 disposes the electrodes 410 at a plurality of radial distances relative to a central longitudinal axis L of the body 404. The electrodes 410 may include an insulated portion 411 and an exposed portion 413. The insulated portion 411 includes an insulative cover positioned between an external surface of the electrode and other contact surfaces. The exposed portion 413 does not include an insulative cover such that the exposed portion can apply energy to tissue when in contact with the tissue.

In some examples, rotation of the rotary drive portion 406 of the body 404 causes the arms 408 to move in concert, disposing the arms 408 at substantially similar distances and positions relative to the central longitudinal axis L of the body 404. In other examples, the arms 408 can have different couplings with the rotary drive portion 406 of the body 404, such that rotation of the rotary drive portion 406 causes the arms 408 to pivot at different rates and distances.

The medical device 402 can be configured with any suitable number of arms 408 and electrodes 410 to perform a given procedure. The arms 408 and electrodes 410 can be arranged in a suitable array for spacing requirements and gauges of a given implementation. For example, the medical device 402 can include two, three, four, or more arms 408 and electrodes 410. In some examples, the medical device 402 can also include a central electrode 410a coupled to the body 404. The central electrode 410a is stationary and would not move with pivoting of the arms 408. As can be understood, however, relative spacing between the central electrode 410a and the electrodes 410 coupled to the arms 408 would vary as the arms 408 were pivoted.

As shown, the arms 408 can have an arcuate configuration to wrap around the body 404 in the retracted state. The arcuate shape allows the medical device 402 to maintain a compact configuration for delivery to the treatment location. Additionally, depending on desired extended states and the number of arms 408, the arms 408 can be sized and configured to partially overlap one another along a circumference of the body 404.

The electrodes 410 can be utilized to deliver energy-based focal treatment in either a monopolar or bipolar fashion (e.g., Radio Frequency ablation (RFA), Electroporation-based energy (pulsed electric field (PEF)/pulsed field ablation (PFA), nanosecond pulsed electric field (nsPEF), high-frequency irreversible electroporation (HFIRE), irreversible electroporation (IRE), combined electroporation and electrolysis (E2), electricchemotherapy (ECT), electrical impedance tomography (EIT), electro-gene-transfer (EGT)), Electrolytic-based energy, or any combination.). For some treatments, at least one of the electrodes 410 can be a cathode and at least one of the electrodes 410 can be an anode while performing an energy treatment. Additionally or alternatively, the two or more electrodes 410 can enable local tissue sensing during a procedure (e.g., before, during, or after energy treatment). The local tissue sensing can provide data regarding the tissue, including, for example, impedance, temperature, pH, etc. Optionally, one or more of the electrodes 410 may have an insulators extending therearound at least partially along a length thereof.

The electrodes 410 can have a uniform configuration (e.g., shape and size) or can have two or more different configurations/characteristics. In some examples, at least one of the electrodes 410 has a solid cross-section (e.g., is not hollow). In some examples, at least one of the electrodes 410 defines a channel or lumen extending therethrough for fluid delivery. The electrodes 410 can be configured to penetrate tissue to perform the penetrating energy treatment. In some examples, at least one of the electrodes 410 is a surface electrode (e.g., has a blunt tip to avoid penetration into tissue). The surface electrode 410 can have a shortened configuration relative to penetrating electrodes. The electrodes 410 can have asymmetric lengths to deliver energy treatment at a variety of tissue depths. The electrodes 410 may include microneedles for larger arrays. The electrodes 410 may each extend along a respective longitudinal axis substantially parallel to each other and substantially parallel to the central longitudinal axis L of the medical device 402.

In any of the examples described herein, one or more of the electrodes 410 can include a sensor extending at least partially therethrough. For example, one or more of the electrodes 410 can include a visual tool, such as an ultrasound device (e.g., a fiber optic ultrasound device) or an optical coherence tomography device.

The medical device 402 can also be configured for cryoablation or microwave ablation. In a cryoablation configuration, one or more of the electrodes 410 have an internal channel for nitrogen circulation. In a microwave ablation configuration, one or more of the electrodes 410 have an antenna provided by two internal conductive components spaced by a suitable insulative material (e.g., Teflon). For example, the antenna can be a triaxial antenna, a choke antenna, or a balun-free antenna.

As shown in FIG. 4A, the medical system 400 includes one or more energy sources (e.g., power supplies) 412 that are electrically connected to the electrodes 410 (e.g., by wires and/or other suitable electrical pathways, such as traces, circuit boards, etc.). A control system 414 for the medical system 400 is operably coupled to the power supply 412 to control the electrical energy delivered to the electrodes 410 of the medical device 402.

An output 416 of a rotary drive 418 (e.g., a drive shaft of a motor, etc.) is coupled to and configured to drive rotation of the rotary drive portion 406 of the body 404 to thereby pivot the arms 408. For example, the control system 414 can be operably coupled to the rotary drive 418 and configured to selectively operate the rotary drive 418 to pivot the arms 408 and dispose the electrodes 410 at a desired distance relative to the central longitudinal axis L of the body 404.

The medical device 402 can be configured as a drop-in probe end effector separate from an instrument 420 or as an end effector on the distal end of an instrument 420. In either implementation, the medical device 402 is disposed distally of the instrument 420 and positioned thereby. The instrument 420 can be a rigid, substantially rigid, or flexible instrument as described herein.

In the drop-in probe configuration, the medical device 402 is inserted or delivered at a target location within a patient and the separate instrument 420 includes an end effector capable of grasping and manipulating the medical device 402, such as an end effector including tool members 318, 319 described above. For a drop-in probe configuration, the medical device 402 can include one or more grasping portions 423 to provide a region for holding and manipulating the medical device 402 with a grasping tool, such as forceps.

With the drop-in probe configuration as shown in FIGS. 4D and 4E, the medical device 402 can include a base 422 coupled to the body 404 sized to receive the rotary drive 418 therein, such that the rotary drive 418 is carried by the medical device 402 and delivered to a treatment location. The base 422 and body 404 can couple together with any suitable configuration, such as press-fit, threading, snap fit, latches, adhesive, welding, and so forth. Additionally, the coupling between the base 422 and body 404 can prevent relative rotation between the components. For example, the base 422 or body 404 can define a non-circular cavity 424 and the other of the base 422 or body 404 can have a complementary plug 426 sized to fit within the cavity 424. The cavity 424 and plug 426 can be polygonal (e.g., triangular, rectangular, pentagonal, hexagonal, etc.), suitable curvilinear shapes, keyed shapes, crosses, and so forth. In other examples, the base 422 and body 404 can be integral with one another as a single piece component. Electrical couplings for the rotary drive 418 and electrodes 410 will extend proximally from the medical device 402 to a power source located at a backend mechanism.

In the elongated instrument configuration as shown in FIG. 4A, the medical device 402 can form a distal end of the instrument 420 as an end effector for the instrument 420. In some embodiments, the instrument 420 may have a shaft with a substantially rigid member. In other embodiments, the instrument 420 may have a flexible shaft, and the instrument 420 may be configured to be inserted through a flexible elongate device, such as the flexible elongate device 202 described above. With the elongated instrument configuration, the medical device 402 can include the base 422 with the rotary drive 418 received therein or the rotary drive 418 can be disposed proximally of the instrument 420 with a torque shaft 428 extending within the instrument 420 to be rotatably coupled between the output 416 of the rotary drive 418 and the rotary drive portion 406 of the medical device 402. For example, the elongated instrument may be coupled to a robotic system, wherein the robotic system contains the rotary drive 418. The robotic system has motor outputs that can be operably coupled to robotic inputs at a backend housing of the elongated instrument to transmit forces and torques to the elongated instrument. The backend housing of the elongated instrument includes a force transmission mechanism (which may be similar to force transmission mechanism 304) coupled to a proximal end of the torque shaft 428 to transmit forces/torques to the distal end of the instrument.

The control system 414 can be configured to operate the rotary drive 418 to dispose the arms 408, and the electrodes 410 coupled thereto, at a plurality of discrete positions (e.g., two, three, four, or more) corresponding to desired electrode 410 spacing relative to one another and the central longitudinal axis L of the body 404. In this instance, a user could select which set spacing is desired and the control system 414 will control the rotary drive 418 accordingly. Alternatively, the control system 414 can track incremental spacing of the electrodes 410 during operation of the rotary drive 418 to dispose the electrodes 410 at any desired spacing within the range of motion of the arms 408.

As shown in FIGS. 4D and 4E, the system 400 can include a movable or removable cover 430 for the tips of the electrodes 410. The cover 430 can be used, for example, to deliver the medical device 402 to a treatment location with the tips of the electrodes 410 concealed. Thereafter, the cover 430 can be removed or moved relative to the electrodes 410 to expose the tips of the electrodes 410 to perform an energy treatment.

In one example, the cover 430 includes channels or apertures 432 sized to slidingly receive the electrodes 410. The channels or apertures 432 can be sized to frictionally engage the electrodes 410 so that inadvertent exposure of the tips is prevented. When pressed, such as by tissue during a treatment, the cover 430 can slide proximally along the electrodes 410 to expose a desired length of electrodes 410 for treatment. If desired, the cover 430 can be biased to conceal the tips of the electrodes 410 by a suitable mechanism, such as a spring or compressible material. In another implementation, the cover 430 can be a cap with the channels/apertures 432 not extending fully through the cover 430. With this configuration, the cover 430 can be pulled distally off of the electrodes 410. The cover 430 can include a tether 434 to ensure that the cover 430 remains connected to the device 402 after being removed from the electrodes 410.

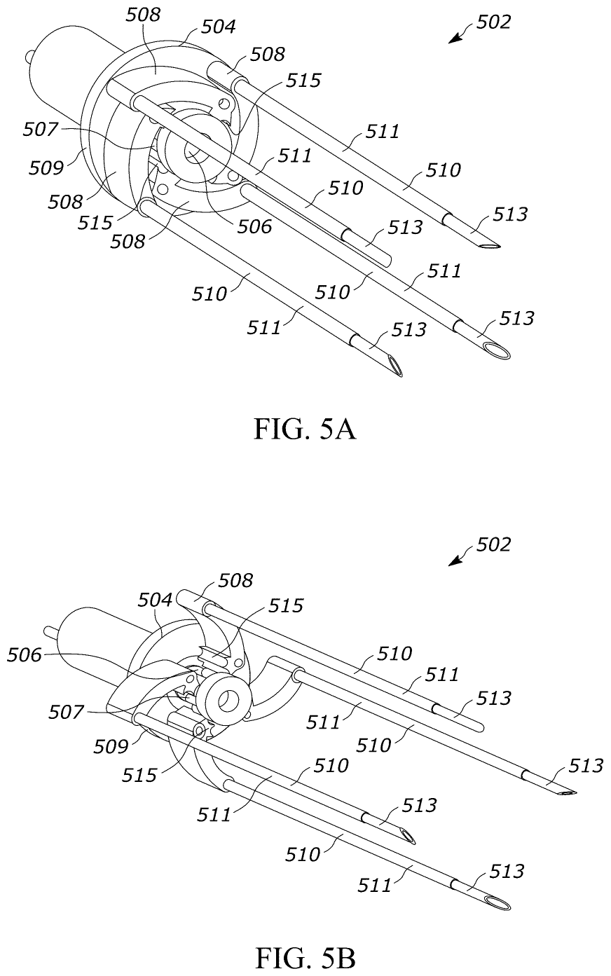

FIGS. 5A and 5B depict a medical device 502 having a body 504 with a rotary drive portion 506 and a plurality of arms 508 pivotably coupled to the body 504. According to some embodiments consistent with FIGS. 4A and 4B, the medical device 502 may correspond to the medical device 402 of the medical system 400.

One example configuration for the rotary drive portion 506 and arms 508 for the medical device 502 having electrodes 510 coupled thereto is shown. The electrodes 510 may include an insulated portion 511 and an exposed portion 513. The insulated portion 511 includes an insulative cover positioned between an external surface of the electrode and other contact surfaces. The exposed portion 513 does not include an insulative cover such that the exposed portion can apply energy to tissue when in contact with the tissue. In this example, the rotary drive portion 506 is an internal post including a gear 507 (e.g., spur gear) having teeth extending radially outwardly at least corresponding to interfaces with each of the arms 508. The gear 507 can have portions disposed along one or more areas of the longitudinal length of the rotary drive portion 506 or can extend the entire exposed length thereof imparting one or more elongate faces. The body 504 includes a stationary portion 509 (e.g., a frame, outer shell, post, etc.) that is stationary with respect to the rotary drive portion 506 and the arms 508 are pivotably mounted to the stationary portion 509. For example, each of the arms 508 can have pin and socket connections or other suitable couplings with the stationary portion 509 at a top and bottom thereof. Each of the arms 508 include an interior gear 515 that meshes with the gear 507 of the rotary drive portion 506, such that rotation of the rotary drive portion 506 drives the arms 508 to pivot with respect to the stationary portion 509 of the body 504. With this configuration, clockwise rotation of the rotary drive portion 506 causes the arms 508 to pivot in a counterclockwise direction and counterclockwise rotation of the rotary drive portion 506 causes the arms 508 to pivot in a clockwise direction. As shown, the arms 508 can have electrodes 510 coupled thereto, so that pivoting of the arms 508 moves the electrodes 510 relative to a central longitudinal axis L of the body 504.

In an alternative configuration, the stationary portion 509 can be an internal post and the rotary drive portion 506 can have an annular configuration disposed at least partially around the stationary portion 509. With this configuration, the arms 508 are pivotably coupled to the stationary portion 509 and the rotary drive portion 506 can include channels for pins of the arms 508 to travel in as the rotary drive portion 506 is pivoted to thereby pivot the arms 508 and the electrodes 510 coupled thereto.

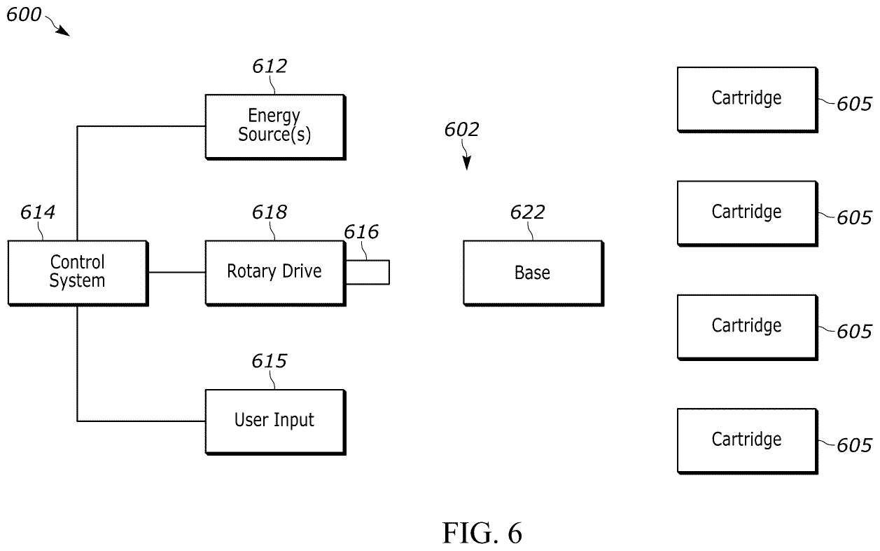

FIG. 6 is a simplified diagram of a medical system 600 including a medical device 602. According to some embodiments consistent with FIGS. 4A-5B, the medical system 600 may correspond to medical system 400 and the medical device 602 may correspond to the medical device 402, 502.

In these examples, the medical system 600 includes a medical device 602 having a plurality of suitable cartridges 605. According to some embodiments consistent with FIGS. 4A-5B, each of the cartridges 605 includes a body (e.g., body 404, 504) with a rotary drive portion (e.g., rotary drive portion 406, 506), a plurality of arms (e.g., 408, 508) pivotably coupled to the body, and electrodes (e.g., 410, 510) coupled to each of the arms. Each arm is pivotable to dispose the electrode coupled thereto at a plurality of distances relative to the central longitudinal axis L of the medical device 602. The medical system 600 also includes a base 622 configured to releasably couple to each of the cartridges 605.

The cartridge 605 have at least one characteristic different from one another. This allows a user to select a desired configuration for a particular treatment. In some examples, the characteristics of the cartridges 605 can include one or more of: number of arms 608/electrodes 610, a central electrode, total length of electrodes 610, length of the exposed portion (e.g., exposed portion 413, 513) of the electrodes 610, length of the insulated portion (e.g., insulated portion 411, 511) of the electrodes 610, spacing of electrodes 610, or type of electrodes 610 (e.g., hollow for fluid delivery, solid cross-section, surface treatment, etc.).

As shown, the medical system 600 further includes a rotary drive 618 (e.g., a motor) having a drive output 616 configured to couple with the rotary drive portion 606 of the body 604 when the cartridge 605 is coupled to the base 622. For example, the coupling can be similar to that described above with respect to the rotary drive 418 and the rotary drive portion 406 (e.g., directly coupled or indirectly coupled via a torque shaft).

The medical system 600 includes one or more energy sources (e.g., power supplies) 612 that are electrically connected to the electrodes 610 (e.g., by wires and/or other suitable electrical pathways, such as traces, circuit boards, etc.) and the rotary drive 618. A control system 614 for the medical system 600 is operably coupled to the energy source(s) 612 to control the electrical energy delivered to one or more of the electrodes 610 pursuant to an energy treatment and to control the electrical energy delivered to the rotary drive 618. For example, the control system 614 is configured to control the supply of electricity from the energy source 612 to operate the rotary drive 618 to pivot the arms 608 to dispose the electrodes 610 coupled thereto at a desired distance relative to the central longitudinal axis L of the cartridge 605. Thereafter, the control system 614 controls the supply of electricity from the energy source 612 to one or more of the electrodes 610 pursuant to an energy treatment.

In some examples, the medical system 600 is configured to identify a selected one of the cartridges 605 to be used for an energy treatment. This identification enables the control system 614 to identify and utilize the particular characteristics of the selected cartridge 605. The identification by the control system 614 of the selected cartridge 605 can include the control system 614 determining a set of parameters for the desired energy treatment corresponding to the selected cartridge 605 and supplying energy to the medical device 602 from the energy source 612 to perform the energy treatment based on the set of parameters.

Identification of the cartridge 605 can be made by any suitable mechanism. For example, a user can input identification information into the system 600 via a suitable user input 615 (e.g., keyboard, touch screen, scanner, mouse, voice recognition, etc.), the cartridge 605 can include a radio frequency identification (RFID) tag configured to be read by the system 600, the cartridge 605 and system 600 can include components (e.g., transmitters, receivers, etc.) to enable wireless, (e.g., near field communication (NFC), Bluetooth, WiFi, and so forth).

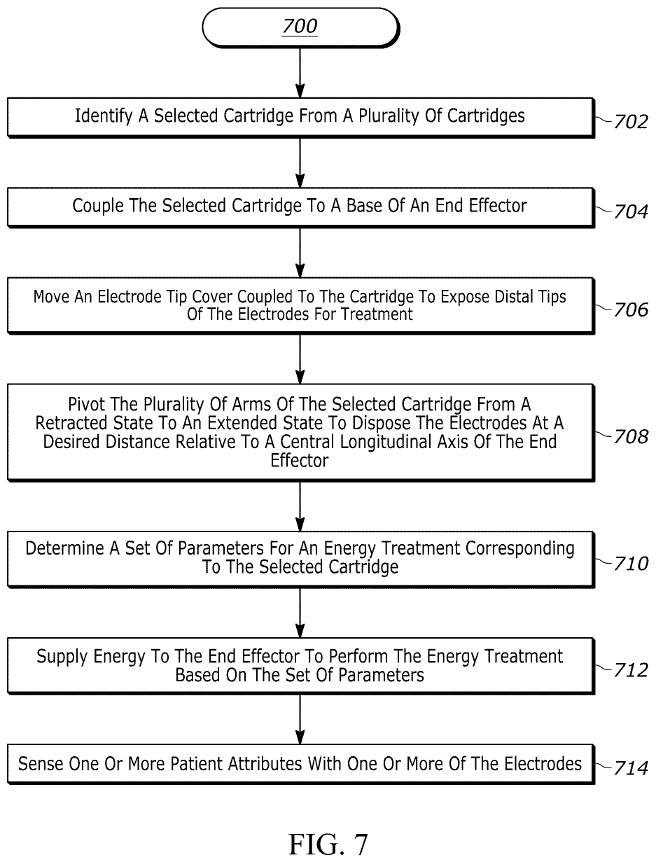

FIG. 7 illustrates a method 700 for energy treatment according to some embodiments. The method 700 is illustrated as a set of operations or processes 702 through 714. Not all of the illustrated processes may be performed in all embodiments of the method 700. Additionally, one or more processes that are not expressly illustrated in FIG. 7 may be included before, after, in between, or as part of the processes 702 through 714. Processes may also be performed in different orders. In some embodiments, one or more of the processes 702 through 714 may be implemented, at least in part, in the form of executable code stored on non-transitory, tangible, machine-readable media that when run by one or more processors (e.g., the processors of a controller) may cause the one or more processors to perform one or more of the processes. In one or more embodiments, the processes 702 through 714 may be performed by a controller.

In process 702, a selected cartridge from a plurality of cartridges (e.g., cartridges 605) is identified. The cartridges have at least one characteristic different from one another and are configured to releasably couple to a base of an end effector (e.g., base 622 and medical device 602). The cartridges each have a plurality of arms (e.g., arms 608) and each arm of the plurality of arms has an electrode coupled thereto (e.g., electrodes 610).

In some examples, identifying the selected cartridge from the plurality of cartridges includes receiving, by a control system, an identification of the selected cartridge from the plurality of cartridges. In further examples, receiving the identification of the selected cartridge from the plurality of cartridges by a control system includes wirelessly receiving the identification of the selected cartridge from the plurality of cartridges.

In process 704, the selected cartridge is coupled to the base of the end effector. In process 706, an electrode tip cover (e.g., cover 430) coupled to the cartridge is moved to expose distal tips of the electrodes for treatment. In process 708, the plurality of arms of the selected cartridge are pivoted from a retracted state to an extended state to dispose the electrodes at a desired distance relative to a central longitudinal axis of the end effector. In some examples, pivoting the plurality of arms of the selected cartridge can include controlling operation of a motor (e.g., rotary drive (418, 618) to pivot the plurality of arms and dispose the electrodes at a desired distance relative to the central longitudinal axis of the end effector.

In process 710, a set of parameters for an energy treatment corresponding to the selected cartridge is determined. In some examples, the set of parameters include one or more of: total electrode length, exposed electrode length, electrode spacing, and a number of electrodes. In process 712, energy is supplied to the end effector to perform the energy treatment based on the set of parameters. In some examples, supplying energy to the end effector to perform the energy treatment includes supplying a plurality of predetermined doses of energy to the end effector to perform the energy treatment. In some examples, supplying energy to the end effector to perform the energy treatment includes supplying at least one of bipolar or monopolar energy to the end effector to perform the energy treatment. In process 714, one or more patient attributes are sensed with one or more of the electrodes.

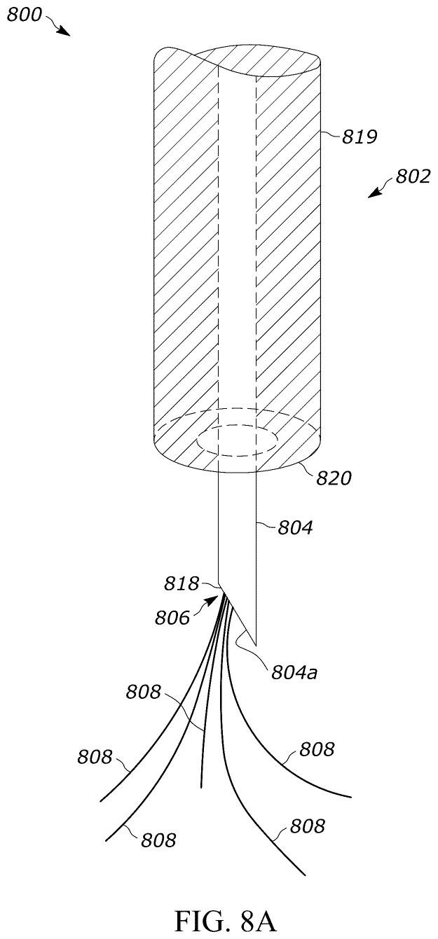

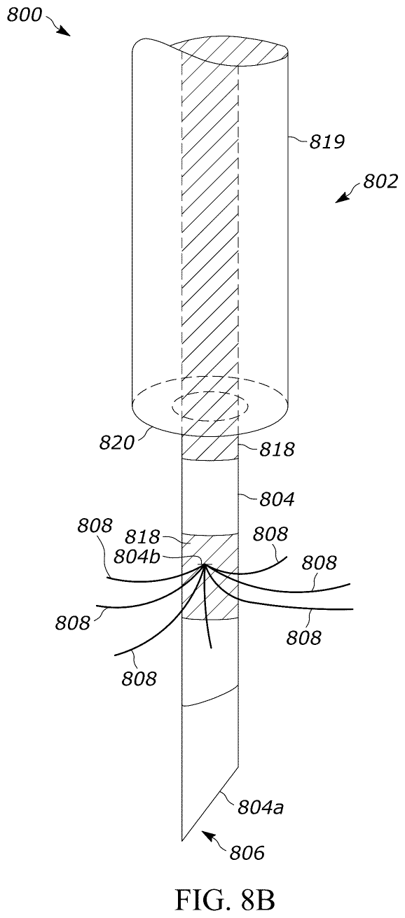

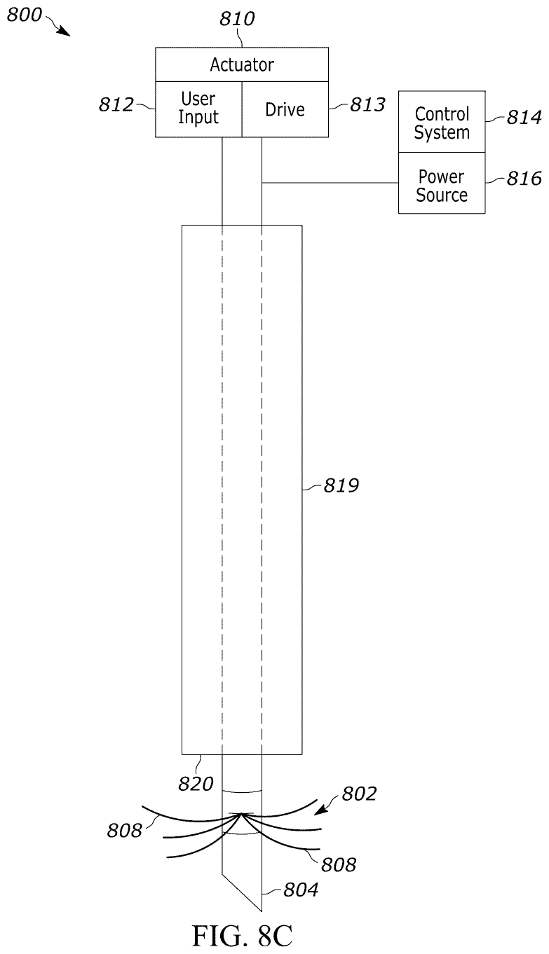

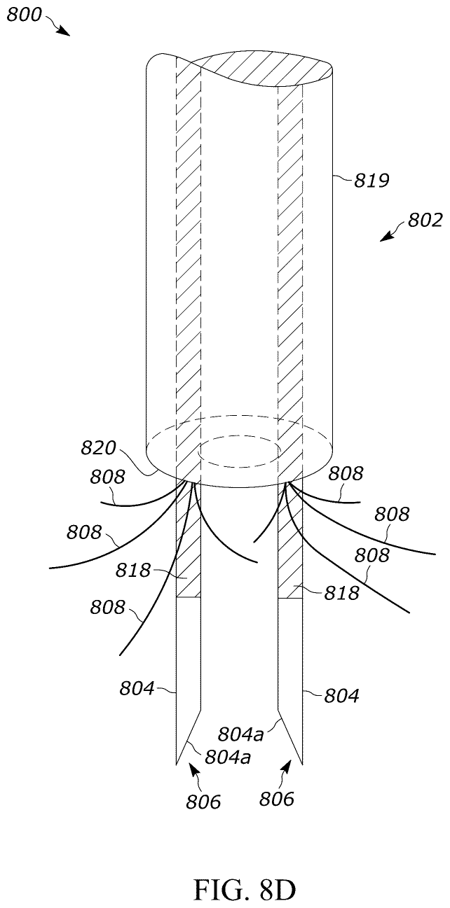

FIGS. 8A-8D show medical systems 800 and components thereof according to some embodiments. The medical systems 800 includes a medical device 802, such as an end effector, capable of performing energy treatment (e.g., penetrating and/or surface energy treatment) with a plurality of electrodes. The energy treatment can include a single dose or can include a plurality of predetermined doses. The electrodes of the medical device 802 include a needle 804 (e.g., a trocar needle) defining one or more lumens 806 and one or more tines 808 received within the lumen(s) 806 of the needle 804. The tines 808 are movable relative to the needle 804 to be deployable into target tissue to provide a larger treatment volume for the medical device 802. The medical device 802 can include any desired number of tines 808 for a particular treatment, such as, one, two, three, four, five, six, seven, eight, or more tines 808. The medical device 802 can be part of a drop-in component (e.g., a tool that can be delivered to a treatment site and grasped/positioned by other surgical instruments to provide treatment). In other versions, the medical device 802 can be an end effector of an elongated surgical instrument, for example an instrument including an elongated torque/drive shaft (rigid or flexible) extending proximally to a backend housing.

The tines 808 are pre-shaped to have an outward angulation (e.g., cant, tilt, slant, etc.) of a desired degree (e.g., between 0 degrees and 45 degrees) or to have a curve, arch, bend, hook, etc. of a desired degree (e.g., between 0 degrees and 180 degrees). In the example shown, the tines 808 have an angulation causing the tines 808 to splay out from the needle 804 during deployment. The angulation can be selected to achieve a desired deployment position within the target tissue. For example, the angulation can be 45 degrees, 90 degrees, 180 degrees, and so forth.

In some examples, the tines 808 can have the same angulation. In other examples, the tines 808 can have different angulations. For example, every other tine 808 could have a similar angulation to distribute the tines 808 at different depths. In another example, tines 808 on one side of the needle 804 could have a first angulation and tines 808 on another side of the needle 804 could have a second angulation. It will be understood that angulations can also be selected to achieve a desired distribution within the target tissue, such as to match a size and/or shape of the target tissue. For example, a select number of the tines 808 can be pre-shaped to selectively expand the treatment volume in desired directions, while the remaining tines 808 are not deployed or have a straighter (e.g., more parallel to a longitudinal axis of the needle 804) to provide directed volume expansion. This can advantageously be used to avoid harming adjacent critical structures.

FIG. 8A illustrates a first deployment configuration for the tines 808 from the needle 804. In this example, the tines 808 deploy through a distal opening 804a of the needle 804. FIG. 8B illustrates a second deployment configuration for the tines 808 from the needle 804. In this example, the tines 808 deploy from openings 804b (e.g., slot openings) defined in the sidewall of the needle 804.

In either configuration, the needle 804 can define one central lumen for simultaneous deployment of the tines 808 or can define lumens for each tine 808 or subsets of tines 808 for selective deployment.

As shown in FIG. 8C, the device 802 further includes an actuator 810 operably coupled to the tines 808 to drive deployment of the tines 808 from the needle 804 and into target tissue. The actuator 810 includes a user input 812 configured to be manipulated by a user to activate the actuator 810. The user input 812 may be located at a proximal end region of an instrument including the device 802 (e.g., at a backend housing of the instrument). Additionally or alternatively, the user input 812 may be at an operator input system (such as operator input system 106). The actuator 810 can include any suitable drive mechanism to move the tines 808. For example, the user input 812 can be operable to allow a user to manually push the tines 808 for deployment, such as with one or more shafts extending proximally from the medical device 802. In another example, the actuator 810 can include a drive 813, such as a spring-biased mechanism, an electro-magnet mechanism, motor, etc., that drives deployment of the tines 808 in response to activation by the user.

Exposed surfaces of the needle 804 and the tines 808 can be utilized to deliver an energy treatment. For example, as shown in FIG. 8C, the medical system 800 includes a control system 814 and a power source 816 electrically coupled to the needle 804 and the tines 808. The control system 814 is configured to deliver energy to the medical device 802 according to a treatment protocol (e.g., a single dose or a plurality of doses).

For some implementations, the needle 804 and the tines 808 can have the same polarity for a monopolar treatment. In other implementations, the needle 804 and the tines 808 can have a different polarity to deliver a bipolar treatment. As such, the needle 804 can be an anode and the tines 808 can be cathodes. Advantageously, it has been found that an asymmetric surface area for the anode and the cathode for a bipolar energy treatment can provide some advantages for treatment and the relatively larger needle 804 provides a larger surface area than the combined surface of the relatively smaller tines 808. Of course, the needle 804 could alternatively be a cathode and the tines 808 can be anodes or the needle 804 can have the same polarity as a subset of the tines 808.

With this configuration, a user inserts the needle 804 to a desired depth within the target tissue. Thereafter, the user activates the actuator 810 to deploy the tines 808 into the target tissue and causes the control system 814 to provide power to the needle 804 and tines 808 according to an energy treatment protocol. The energy treatment protocol can include activating the all of the tines 808 simultaneously along with the needle 804.

In some examples, the needle 804 and/or the tines 808 include insulated portions 818 to provide an insulative cover positioned along an external surface to prevent electrical engagement with other contact surfaces. For example, the needle 804 can include an insulated portion 818 around the distal opening 804a or the side openings 804b to insulate the needle 804 from the tines 808. Exposed portions of the needle 804 and/or tines 808 that do not include an insulative cover can apply energy to tissue when in contact with the tissue.

As shown in FIG. 8C, the medical device 802 can include a body 819 to which the needle 804 is coupled or passes through at a treatment location. In one example, the body 819 can be the body of an end effector having the needle 804 mounted thereto. In another example, the body 819 is an elongate device utilized to guide the needle 804 to the treatment location through a lumen defined thereby. An end surface 820 of the body 819 facing target tissue during a procedure can advantageously be utilized as a surface electrode, either as an anode or cathode, in combination with the needle 804 and tines 808 for a procedure. Pursuant to this, the body 819 can be made of a suitable electrode material with an insulated covering to expose to the end surface 820, or the body 819 can include an electrode member/plate at a distal end thereof to provide the end surface 820.

With this configuration, a user inserts the needle 804 into the target tissue until the end surface 820 abuts the target tissue or inserts the needle 804 to a desired depth within the target tissue and advances the body 819 relative to the needle 804 until the end surface 820 abuts the target tissue. Thereafter, the user activates the actuator 810 to deploy the tines 808 into the target tissue and causes the control system 814 to provide power to the needle 804, tines 808, and end surface 820 according to an energy treatment protocol. The polarity of the components can have any desired configuration. For example, the tines 808 can have the same polarity and the needle 804 and end surface 820 can have the opposite polarity.

Another example for the medical device 802 is shown in FIG. 8D. In this example, the medical device 802 includes two needles 804, each of which defines one or more lumens 806 and has one or more tines 808 received within the lumen(s) 806 of each needle 804. The tines 808 are movable relative to the respective needle 804 to be deployable into target tissue to provide a larger treatment volume for the medical device 802. The medical device 802 can include any desired number of tines 808 for each needle 804, such as, one, two, three, four, five, six, seven, eight, or more tines 808.

The needle(s) 804 and tines 808 can be any suitable conducting metal, such as stainless steel, Titanium alloy, Tungsten, gold, and so forth. The medical device 802 can have a diameter suitable to be delivered through a flexible or rigid elongate device. The needle 804 can have any suitable gauge, such as 16 GA or less.

One or more components of the embodiments discussed in this disclosure, such as control system 112, 414, 614 may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Various general-purpose computer systems may be used to perform one or more processes, methods, or functionalities described herein. Additionally or alternatively, various specialized computer systems may be used to perform one or more processes, methods, or functionalities described herein. In addition, a variety of programming languages may be used to implement one or more of the processes, methods, or functionalities described herein.

While certain embodiments and examples have been described above and shown in the accompanying drawings, it is to be understood that such embodiments and examples are merely illustrative and are not limited to the specific constructions and arrangements shown and described, since various other alternatives, modifications, and equivalents will be appreciated by those with ordinary skill in the art.

What is claimed is:

1. A medical device comprising:
a body including a rotary drive portion;
a plurality of arms pivotably coupled to the body to be movable by rotation of the rotary drive portion of the body between a retracted state extending along the body and an extended state at least partially extending outwardly from the body relative to the retracted state; and
a plurality of electrodes, each of the plurality of arms having one of the plurality of electrodes coupled thereto.

2. The medical device of claim 1, wherein the plurality of arms abut against the body in the retracted state.

3. The medical device of claim 1, further comprising a rotary drive output coupled to and configured to drive rotation of the rotary drive portion of the body.

4. The medical device of claim 3, further comprising:
an end effector base coupled to the body; and
a rotary drive received within the end effector base, the rotary drive output comprising an output shaft of the rotary drive.

5. The medical device of claim 3, further comprising:
an elongate instrument; and
a torque shaft extending within the elongate instrument, wherein the body is coupled to a distal end of the elongate instrument and the torque shaft is rotatably coupled between the rotary drive output and the rotary drive portion of the body.

6. The medical device of claim 1, wherein the rotary drive portion comprises a drive gear and interior ends of the respective plurality of arms comprise a driven gear configured to mesh with and be driven by drive gear.

7. The medical device of claim 1, wherein the plurality of arms are pivotable to position the plurality of electrodes at two or more predetermined positions.

8. The medical device of claim 1, wherein at least one of the plurality of electrodes defines a lumen extending therethrough for fluid delivery.

9. The medical device of claim 1, wherein the plurality of electrodes have asymmetric lengths.

10. The medical device of claim 1, further comprising a central electrode coupled to the body radially inwardly of the plurality of electrodes coupled to the plurality of arms.

11. The medical device of claim 1, further comprising a cover movable to selectively expose distal tips of the plurality of electrodes.

12. A medical system comprising:
an end effector having a central longitudinal axis, the end effector comprising:
a base;
a plurality of cartridges configured to be releasably attached to the base, each of the plurality of cartridges including a plurality of arms, each arm of the plurality of arms having an electrode coupled thereto and being pivotable to dispose the electrode at a plurality of distances relative to the central longitudinal axis of the end effector, the plurality of cartridges having at least one characteristic different from one another;
an energy source;
a control system configured to:
identify a selected one of the plurality of cartridges to be used for an energy treatment;
determine a set of parameters for the energy treatment corresponding to the selected one of the plurality of cartridges; and
supply energy to the end effector from the energy source to perform the energy treatment based on the set of parameters.

13. The medical system of claim 12, wherein the set of parameters comprise one or more of: total length of the electrodes, length of an exposed portion of the electrodes, length of an insulated portion of the electrodes, electrode spacing, and a number of electrodes.

14. The medical system of claim 12, wherein the control system configured to identify the selected one of the plurality of cartridges comprises receiving, by the control system, an identification of the selected one of the plurality of cartridges.

15. The medical system of claim 12, further comprising a motor, each cartridge including a drive portion coupled to the motor when attached to the base of the end effector to be rotated thereby, and wherein the control system is configured to control operation of the motor to pivot the plurality of arms and dispose the electrodes at a desired distance relative to the central longitudinal axis of the cartridge.

16. The medical system of claim 12, wherein the end effector comprises a drop-in probe.

17. The medical system of claim 12, wherein the end effector comprises a distal end of an elongate instrument.

18. The medical system of claim 12, wherein the control system is further configured to use one or more of the electrodes for sensing one or more patient attributes.

19. An energy treatment method comprising:
identifying a selected cartridge from a plurality of cartridges having at least one characteristic different from one another and configured to releasably couple to a base of an end effector, the plurality of cartridges each having a plurality of arms, each arm of the plurality of arms having an electrode coupled thereto;
coupling the selected cartridge to the base of the end effector;
pivoting the plurality of arms of the selected cartridge from a retracted state to an extended state to dispose the electrodes at a desired distance relative to a central longitudinal axis of the end effector;
determining, by a control system, a set of parameters for an energy treatment corresponding to the selected cartridge; and
supplying, by the control system, energy to the end effector to perform the energy treatment based on the set of parameters.

20. The method of claim 19, wherein the set of parameters comprise one or more of: total length of the electrodes, length of an exposed portion of the electrodes, length of an insulated portion of the electrodes, electrode spacing, and a number of electrodes.

21. The method of claim 19, wherein identifying the selected cartridge from the plurality of cartridges comprises receiving, by a control system, an identification of the selected cartridge from the plurality of cartridges.
